(19) United States
(12) Patent Application Publication
(10) Pub. No.: US 2017/0027538 A1
(43) Pub. Date: Feb. 2, 2017

(54) MOBILE DATA GLASSES AND METHOD FOR COMMUNICATION BETWEEN A MEDICAL NETWORK AND MEDICAL STAFF USING MOBILE DATA GLASSES

(71) Applicant: Siemens Healthcare GmbH, Erlangen (DE)

(72) Inventors: Katharina Aschoff, Erlangen (DE); Anja Jaeger, Fuerth (DE); Stephan Nufer, Erlangen (DE); Thorsten Speckner, Erlangen (DE); Jens Thoene, Nuernberg (DE)

(73) Assignee: Siemens Healthcare GmbH, Erlangen (DE)

(21) Appl. No.: 15/223,128

(22) Filed: Jul. 29, 2016

(30) Foreign Application Priority Data
Jul. 29, 2015 (DE) 102015214350.3

(51) Int. Cl. A61B 6/00; A61B 6/03; G02B 27/01; A61B 5/055

(52) U.S. Cl. CPC A61B 6/563; A61B 5/055; A61B 6/032; A61B 6/037; G02B 27/017; A61B 2090/502

(57) ABSTRACT

In a method for communication between a user and a medical network into which at least one medical imaging device is integrated, using mobile data glasses, the mobile data glasses are connected to the medical network by a network unit, and a medical examination to be conducted on a patient is selected from a database by a communication unit. At least one item of examination-relevant information of the medical examination to be conducted on the patient is also selected by the communication unit. The at least one item of examination-relevant information is displayed at the mobile data glasses.

### BACKGROUND OF THE INVENTION

Field of the Invention

The present invention concerns communication between a user and a medical network into which at least one medical imaging device is integrated, by the use of mobile data glasses. The present invention further concerns mobile data glasses that have a network unit and a communication unit that make the mobile data glasses useable for such communication.

Description of the Prior Art

In order for a medical imaging examination, in particular a magnetic resonance examination or a computed tomography examination, to be conducted on a patient, it is necessary to make preparations for the examination and/or to prepare the patient therefor. The preparation can involve an entry and/or an acknowledgment of patient parameters and/or patient information. Furthermore, the preparation can involve an exact placement and/or positioning of the patient on a patient support device and/or a docking of the patient support device to a medical imaging device. The arranging of an ancillary unit, for example an ECG unit, an injector unit, etc. in proximity to the patient can also be affected by the preparation of the medical examination. For a magnetic resonance examination it may additionally be necessary for local radiofrequency antenna units tuned to the magnetic resonance examination to be applied to the patient.

In order to carry out such preparation steps it may be necessary for a user, such as a member of the medical staff overseeing the medical imaging examination, to receive forms of assistance (aids) and/or instructions that are visually presented to the user via a display integrated in the medical imaging device and/or a monitor integrated in the medical imaging device. For communication between the medical imaging device and the user of patient-specific data it is furthermore necessary to complete a registration of the user because at least some of the patient-specific data are subject to strict data protection requirements.

### SUMMARY OF THE INVENTION

An object of the present invention is to provide simple and direct support for a user during the preparation of a medical examination that is to be conducted on a patient.

The above object is achieved in accordance with the invention by a method for communication between a user and a medical network into which a medical imaging device is integrated, and by the use of mobile data glasses. The method has the following steps. The mobile data glasses are connected to the medical network via a network unit. A medical examination to be conducted on a patient is selected from a database via a communication unit. At least one item of examination-relevant information of the medical examination to be conducted on the patient is selected via the communication unit. The at least one item of examination-relevant information is displayed to the user by the mobile data glasses.

As used herein, “data glasses” means a pair of eyeglasses with which a user is able to see regions that are arranged on the side of the eyeglasses facing away from the user, and at which data and/or information is displayed to the user. In this case data are and/or information is preferably visualized on and/or projected onto at least one of the two lenses of the eyeglasses, so that the data and/or information can be displayed and/or visualized directly in a visual range and/or a field of view of the user. For this purpose, the mobile data glasses preferably include the communication unit as an integrated component thereof, and the communication unit includes a visualization unit for visualizing and/or projecting the data and/or information. Such mobile data glasses thus are specially designed for performing the method independently of the location of the user who is wearing the mobile data glasses. The mobile data glasses are preferably connected to the medical network via a WLAN connection.

A medical network as used herein means multiple medical units and/or medical devices, that are or can be connected to one another for the purpose of data exchange. In this case the medical units and/or medical devices can include at least one medical database and/or at least one medical imaging device and/or at least one control computer and/or at least one communication unit, etc. The medical network can be arranged within a hospital, for example, in which case access to patient data and/or examination data etc. can be accomplished via the medical network. Preferably, the at least one database is a database in data for conducting which medical examinations are stored, and is integrated into the medical network.

The medical examination to be conducted on the patient preferably is a medical imaging examination, in particular a magnetic resonance examination or a computed tomography examination and/or an angiography examination and/or a PET examination (positron emission tomography examination), etc. The inventive method can be used particularly advantageously in medical imaging examinations, because in this context a particularly elaborate and time-consuming and, at least to some degree, complex preparation of the patient may be necessary. In this situation, the inventive method enables the user to continue to have both hands free for the preparation of the patient and at the same time to be able to receive visual information and/or a visual aid and/or a visual working instruction via the mobile data glasses.

The communication unit can be incorporated in the mobile data glasses or alternatively in a unit embodied separately from the mobile data glasses. Access to the medical network by the mobile data glasses, and/or vice versa, is accomplished via the communication unit.

With this embodiment of the invention, simple and direct support for a user during a preparation of a medical examination, in particular a medical imaging examination, is provided. Examination-relevant information can in this case be displayed and/or visualized at all times for the user, such as a member of the medical operating staff overseeing the medical imaging examination, in a field of view and/or in a visual range of the user. Thus a change in viewing direction, and consequently a turning of the head by the user, in order to assimilate the examination-relevant information is not necessary. Moreover, the operational procedure of the user can be improved, in particular simplified, and performed in a time-saving manner because information and/or aids and/or working instructions can be shown directly to the user and can also be directly assimilated by the latter. Furthermore, confidential information that is covered by special data protection regulations can also be presented to the user, because this confidential information is visible only to the user, and the confidential information is thereby prevented from being seen by third party, such as an unauthorized person.

The connection of the mobile data glasses to the medical network can be implemented wirelessly. This affords further freedom of movement to the user of the mobile data glasses.

In another embodiment of the invention, the medical examination is selected automatically. For example, the selection of the medical examination to be conducted on the patient can be specified and/or effected automatically by the communication unit on the basis of patient-specific parameters that are acquired during a patient registration.

Alternatively or in addition, the medical examination can be selected by an input made by the user. In this case the input can be made as a voice input, so that the user continues to have both hands available for preparation of the patient for the medical examination. For this purpose, the communication unit is or has a voice input unit, such as a microphone.

Furthermore, the input by the user can be accomplished by an eye-tracking method, in which case the communication unit has an acquisition unit for detecting eye movement of the user. Moreover, the communication unit then has software and/or computer programs configured for generating information relating to an eye movement of the user on the basis of the data acquired by the acquisition unit. This also allows an advantageous input, not visible to third parties, in the case of data, in particular patient-related data, that are subject to stringent security criteria and/or data protection regulations.

In a further embodiment of the invention, the at least one item of examination-relevant information is selected automatically by the communication unit on the basis of the selected medical examination to be conducted on the patient. This enables the examination-relevant information to be provided in a simple and time-saving manner. The examination-relevant information can be provided directly for display to the user in this way.

In a further embodiment of the invention, the at least one item of examination-relevant information includes at least one aid and/or at least one working instruction for the medical examination to the user. This enables the aid and/or working instruction to be projected as a pop-up to the user directly in a visual range and/or a field of view, in particular a focus range. An active and time-consuming invocation of aids and/or working instructions thus can be dispensed with. The at least one aid and/or the at least one working instruction can be, for example, positioning information for a local radiofrequency antenna unit for a medical imaging examination to be conducted on the patient. The at least one aid and/or the at least one working instruction can also be, for example, a selection of a local radiofrequency antenna unit that is relevant for the medical imaging examination, in particular the magnetic resonance examination.

Furthermore, the at least one item of examination-relevant information can be or include patient-specific information. In this way patient-specific information can be displayed and/or projected as a pop-up directly to the user during an operational procedure, in particular during a preparation of the patient for the medical examination and/or in the course of attending to the patient during the medical imaging examination, and consequently can be directly assimilated by the user. Moreover, by virtue of a visualization by the mobile data glasses, information of this type can be displayed and/or projected as a pop-up so that it is visible only to the user and such information can be prevented from being visible to unauthorized third parties.

Furthermore, the at least one item of examination-relevant information can be status information relating to a medical imaging device by which the medical examination is being performed, as a result of which a user can be kept informed at any time about a current system status. For example, this enables the user to obtain information about when an examination by the medical imaging device can begin and/or to obtain information about how long the medical examination by the medical imaging device still has to run.

Furthermore, the at least one item of examination-relevant information can comprise information relating to a termination of the medical examination. In this way the user can already obtain information that represents further planning, in particular a further plan in relation to further examinations to be conducted on the patient and/or further plans in relation to the medical imaging device. This also supports time-optimized planning of an operational procedure of the user.

In an embodiment of the invention environmental data of the user can be acquired by an acquisition unit of the mobile data glasses. This enables a context-dependent selection of examination-relevant information and also a context-dependent output of information to the user. For example, the user can be alerted in this way to acute risks to the patient. Moreover, working instructions and/or aids that are displayed to the user by means of the mobile data glasses can also be selected and/or adapted with respect to a current situation of the patient and/or of the user. Furthermore, the acquisition of the environmental data by the acquisition unit of the mobile data glasses enables an automated acquisition of information and/or detection of situations which allow an automated evaluation and consequently a subjective evaluation and/or assessment of the situation by the user can be prevented. In this way errors in assessing a situation can be reduced. The environmental data of the user encompasses a field of view and/or a visual range, in particular a focus range, of the user. For example, the environmental data of the user can be include the patient prior to the medical examination or the patient during the medical examination. Preferably, the acquisition unit is a camera so that a particularly affordable acquisition unit can be provided for the mobile data glasses.

The at least one item of examination-relevant information is generated on the basis of the acquired environmental data of the user. This enables the at least one item of examination-relevant information to be adapted to match a current situation and/or to be generated as a function of a current situation and said examination-relevant information to be displayed and/or projected as a pop-up directly and/or immediately to the user. The examination-relevant information can be generated by the communication unit in an automated manner on the basis of the acquired environmental data. This permits an objective acquisition and evaluation of the acquired environmental data in which all situations detected in the acquisition region can be taken into account in the generation and/or determination of the examination-specific information. The at least one generated item of examination-relevant information can in this case comprise an aid and/or a working instruction. This enables a display and/or a pop-up projection to provide visual support to the user during a current operating step. During a positioning of local radiofrequency antenna units for a pending magnetic resonance examination, for example, positioning information for the local radiofrequency antenna unit can be displayed directly to the user as further examination-relevant information by the mobile data glasses.

In a further embodiment of the invention, the at least one item of examination-relevant information includes safety information. This allows the user to be alerted directly and immediately to acute safety problems. For example, the safety information can concern loose-hanging cables, which can lead to the cables being damaged when the patient is introduced into a patient receiving zone of a medical imaging device. Furthermore, the safety information can also concern uninserted plug-in connectors for ancillary units that are necessary for the medical examination, for example an ECG unit and/or an injection unit. Moreover, the safety information can also comprise patient-specific safety information, such as an arranging of the extremities of the patient to form a closed loop for example, which in particular in the case of a magnetic resonance examination can lead to a severe heating of the patient. Alternatively or in addition, the safety information can include further information considered beneficial to those skilled in the art.

In a further embodiment of the invention, a field of view of the user can furthermore be detected and the at least one item of examination-relevant information determined as a function of the detected field of view of the user. This enables a direct display and/or pop-up projection of information which currently concerns the user and/or with which the user is currently dealing. For example, aids for an operating step can in this way be immediately displayed and/or projected as a pop-up to the user while the operating step is being performed by the user.

The invention furthermore relates to mobile data glasses that are designed for performing the method for communication between a user and a medical network into which at least one medical imaging device is integrated. Such mobile data glasses have a network unit configured for connecting the mobile data glasses to the medical network, a communication unit configured to select a medical examination to be conducted on a patient from a database, and further configured for selecting at least one item of examination-relevant information of the medical examination to be conducted on the patient, and a visualization unit at which the at least one item of examination-relevant information is displayed or visualized.

The mobile data glasses in accordance with the invention afford simple and direct support for a user during a preparation of a medical examination, in particular a medical imaging examination. The examination-relevant information can be displayed and/or visualized at all times for the user, in particular a member of the medical operating staff overseeing the medical imaging examination, in a field of view and/or in a visual range, in particular a focus range, of the user. A change in viewing direction and consequently a turning of the head by the user in order to assimilate the examination-relevant information thus can be avoided. Moreover, the operational procedure of the user can be improved, in particular simplified, and performed in a time-saving manner because information and/or aids and/or working instructions can be output directly to the user and can also be directly assimilated by the latter. Furthermore, confidential information that is covered by special data protection regulations can also be provided to the user, because the confidential information is visible only to the user and is not able to be seen by a third party, in particular an unauthorized person.

The advantages of the inventive mobile data glasses substantially correspond to the advantages of the inventive method for communication between a user and a medical network into which at least one medical imaging device is integrated as explained above. Features, advantages and alternative embodiments noted with regard to the method apply to the mobile data glasses as well.

In a further embodiment of the mobile data glasses, the mobile data glasses have an acquisition unit by which environmental data of the user can be acquired. This enables a context-dependent selection of examination-relevant information and also a context-dependent output of information to the user. For example, the user can be alerted in this way to acute and/or real risks to the patient. Moreover, working instructions and/or aids that are displayed to the user by the mobile data glasses with respect to a current situation of the patient and/or of the user can also be selected and/or adapted. Furthermore, the acquisition of the environmental data by the acquisition unit of the mobile data glasses can support an automated acquisition of information and/or detection of situations which enable an automated evaluation and consequently a subjective evaluation and/or assessment of a situation by the user can be prevented. In this way errors in assessing a situation can be reduced. The environmental data of the user encompasses a field of view and/or a visual range, in particular a focus range, of the user. For example, the environmental data of the user concerns a patient prior to the medical examination or the patient during the medical examination. Preferably, the acquisition unit is a camera so that a particularly affordable acquisition unit can be provided for the mobile data glasses.

The invention furthermore encompasses a non-transitory, computer-readable data storage medium encoded with programming instructions (program code) that can be loaded directly into a memory of a programmable communication unit of a pair of mobile data glasses. The program code cause a computer or distributed computers in the network implement the inventive method when the program code is executed. The computer program code may requires peripherals, e.g. libraries and help functions, in order to realize the various embodiments of the method. The program code may be source code that still needs to be compiled and linked or which only has to be interpreted, or an executable software code that only needs to be loaded into a corresponding computer in order to be executed.

### BRIEF DESCRIPTION OF THE DRAWINGS

FIG. 1 schematically illustrates an inventive medical network.

FIG. 2 schematically shows a pair of mobile data glasses in accordance with the invention.

FIG. 3 is a flowchart of an embodiment of the inventive method for communication between a user and a medical network into which at least one medical imaging device is integrated, using mobile data glasses.

FIG. 4 schematic shows a pop-up projection at the mobile data glasses.

FIG. 5 shows, another embodiment of the inventive method.

### DESCRIPTION OF THE PREFERRED EMBODIMENTS

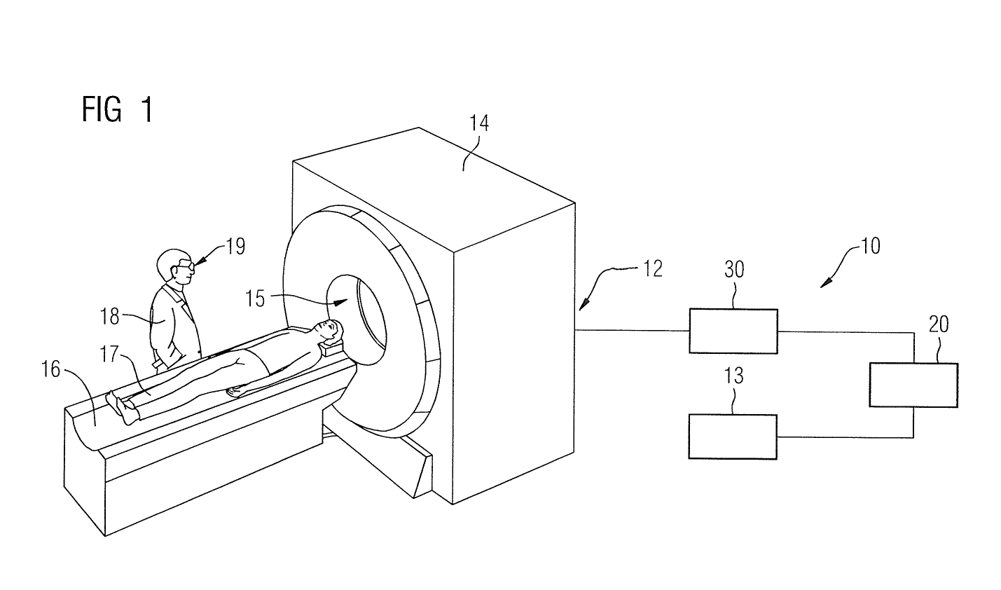

FIG. 1 schematically illustrates a medical network 10. At least one medical imaging device is integrated into the medical network 10. In the exemplary embodiment, a magnetic resonance device 12 is integrated into the medical network 10. In principle, a medical imaging device that is embodied differently to a magnetic resonance device 12, such as a computed tomography device, for example, or indeed two or more medical imaging devices 11, can also be integrated into the medical network 10.

The medical network 10 additionally has a database 13 in which patient information and/or examination information is stored. The medical network 10 can also have further units considered beneficial by those skilled in the art. In the exemplary embodiment the medical network is formed by a hospital network. Alternatively, the medical network 10 can be formed by a further network.

The magnetic resonance device 12 has a scanner 14 that encloses a cylinder-shaped patient receiving zone 15 of the magnetic resonance device 12. The magnetic resonance device 12 additionally has a patient support device 16 on which a patient is supported and positioned for the purpose of a medical imaging examination, in particular a magnetic resonance examination. A system control computer 30 of the magnetic resonance device 12 is configured for controlling the magnetic resonance device 12 and is integrated in the medical network 10.

For a medical examination, in particular a magnetic resonance examination, to be conducted on the patient 17 and/or a preparation of the patient 17 for the medical examination, a user 18 communicates with the medical network 10 in order, for example, to obtain patient information and/or in order to obtain examination information. For communication with the medical network 10, the medical network 10 has a network interface processor 20 that controls and administers access authorizations to the medical network 10. Furthermore, the user 18 wears a pair of mobile data glasses 18 for communication with the medical network unit 10. The mobile data glasses 19 enable the user 18 not only to see regions that are arranged on the side of the glasses facing away from the user 18, but also to assimilate data and/or information projected as a pop-up and/or displayed on at least one lens of the mobile data glasses 19.

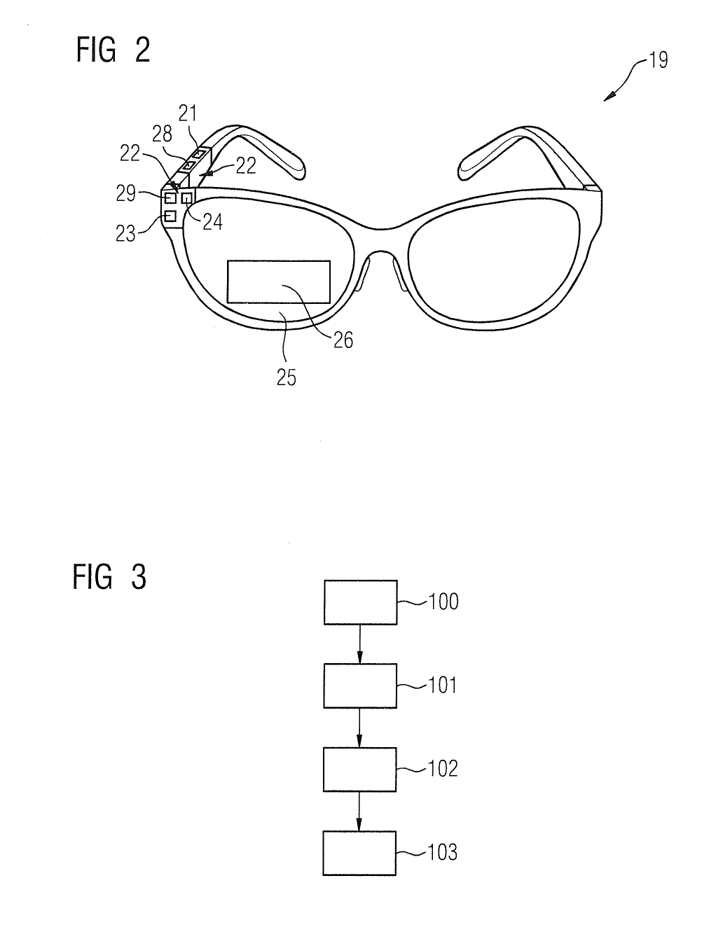

The mobile data glasses 19 are shown schematically in FIG. 2. For communication with the medical network 10, the mobile data glasses 19 have a network interface 21 that is designed to connect the mobile data glasses 19 to the medical network 10. In particular, the network interface 21 is designed to connect the mobile data glasses 19 to the medical network 10 via the network interface processor 20 of the medical network 10, by a WLAN connection. Furthermore, the mobile data glasses 19 have a communication unit 22 that enables a communication between the user 18 and the medical network 10. In particular, the user 18 can also communicate with the medical imaging device 11, in particular the magnetic resonance device 12, by the mobile data glasses 19 and via the medical network 10.

FIG. 3 schematically illustrates an inventive method for communication between the medical network 10 and the user 18 by means of the mobile data glasses 19. In order to perform the method, the communication unit 22 has requisite software and/or computer programs therefor. The software and/or computer programs are stored in a memory (not shown in further detail) of the communication unit 22 and are executed by the mobile data glasses 19 in order to perform the method for communication between the medical network into which at least one medical imaging device 10 is integrated and the user 18. The communication unit 22 thus can be considered as a processor, at least to the extent of having a programmed or programmable chip therein.

In order to perform the method, the user 18 is already wearing the mobile data glasses 18. In a first method step 100, the pair of mobile data glasses 19, in particular the communication unit 22 of the mobile data glasses 19, is connected to the medical network 10 by the network interface 21 of the mobile data glasses 19 and the network interface processor 20 of the medical network 10. In this case the communication unit 22 of the mobile data glasses 19 is connected to the medical network 10 wirelessly and/or without the use of cables by means of the network unit 21 of the mobile data glasses 19 for the purpose of a data exchange. The connection between the communication unit 22 and the medical network 10 can be implemented via WLAN, for example.

In a further method step 101, a medical examination to be conducted on the patient 17 is selected by means of the communication unit 22 from the database 13 arranged or integrated within the medical network 10. The selection can be performed automatically by the communication unit 22, for example when information concerning which patient 17 is to be examined is already present within the communication unit 22, so that it only remains to assign a medical examination, in particular a medical imaging examination, to the patient 17.

Alternatively or in addition, the medical examination can also be selected by an input by the user 18. In this case the input can be accomplished by means of a voice input, such that the user 18 still has both hands free for a preparation of the patient 17 for the medical examination, in particular a medical imaging examination. For this purpose the communication unit 22 has a voice input unit 23. Furthermore, the input by the user 18 can also be effected by means of an eye-tracking method, in which case the communication unit 22 can have for this purpose a sensor unit 24 for detecting an eye movement of the user 18. Moreover, the communication unit 22 can also have software and/or computer programs that are configured for generating information relating to an eye movement of the user 18 on the basis of the acquired data of the sensor unit 24. As support, a selection of possible examinations can in this case also be displayed to the user 18 via the mobile data glasses 18, in particular on a visualization surface 26 of a lens 25 of the mobile data glasses 18.

In the present exemplary embodiment, a medical imaging examination, in particular a magnetic resonance examination to be conducted on the patient 17, is selected in method step 101 of selecting a medical examination. In principle, a selection of a further medical examination, in particular a medical imaging examination, such as a PET examination, for example, or a computed tomography examination, etc., is also conceivable at any time in an alternative embodiment of the invention.

As soon as a medical examination, in particular a medical imaging examination, has been selected, at least one item of examination-relevant information of the medical examination, in particular the magnetic resonance examination, to be conducted on the patient 17 is selected in a further method step 102, in which case the at least one item of examination-relevant information is selected by means of the communication unit 22. In this case the selection of the at least one item of examination-relevant information can be effected automatically and/or autonomously by means of the communication unit 22 on the basis of the selected examination to be conducted on the patient 17.

In the further method step 102 of selecting at least one item of examination-relevant information for the medical examination, in particular the magnetic resonance examination, to be conducted on the patient 17, it is also possible to select a number of items of examination-relevant information or else just one single item of examination-relevant information.

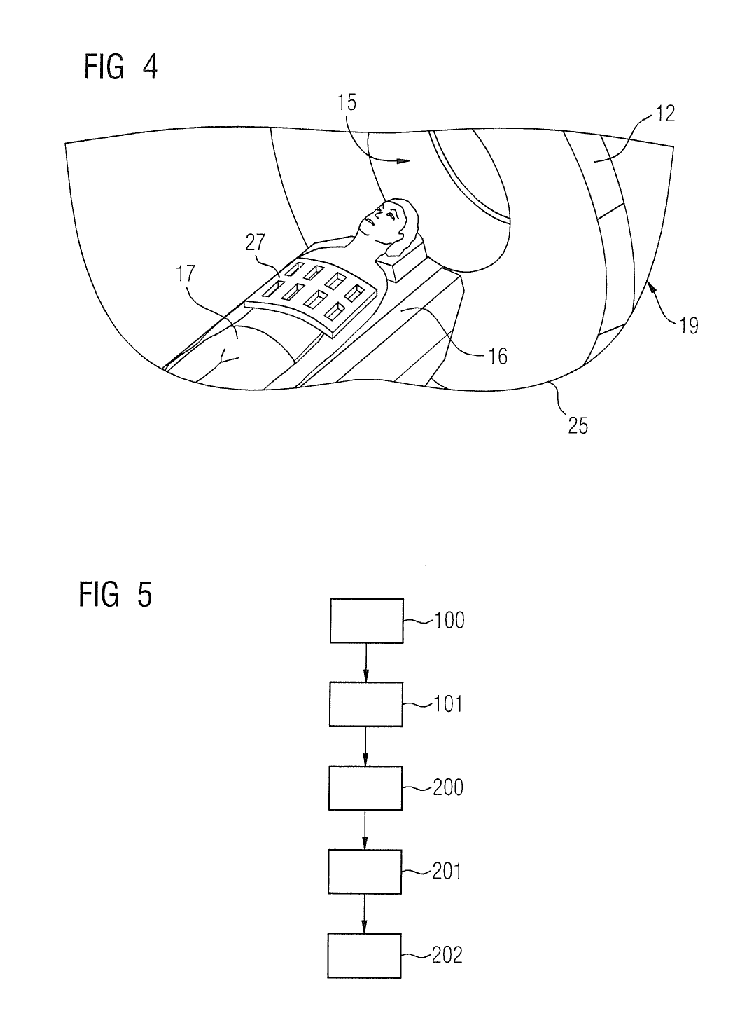

The one item of examination-relevant information or the plurality of items of examination-relevant information can in this case comprise at least one aid and/or at least one working instruction to the user 18 for the selected medical examination, in particular the magnetic resonance examination, to be conducted on the patient 17. The at least one aid and/or the at least one working instruction can be for example positioning information for a local radiofrequency antenna unit for a medical imaging examination to be conducted on the patient 17. In this case the local radiofrequency antenna unit 27 can be shown, for example, as an overlay at a position on the patient 17 (see FIG. 4). Moreover, the at least one aid and/or the at least one working instruction can for example also comprise a selection of the local radiofrequency antenna unit.

The item of examination-relevant information or the multiple items of examination-relevant information can also comprise patient-specific information for the user 18. Patient-specific information of said type can comprise for example a progression of an illness of the patient 17 or personal data of the patient 17. The selection of examination-relevant information which comprises patient-specific information enables a direct display of such information during an operational procedure, in particular during a preparation of the patient 17 for the magnetic resonance examination and/or in the course of attending to the patient 17 during the magnetic resonance examination and consequently can be directly assimilated by the user 18. Moreover, information of this type can be displayed and/or projected as a pop-up so as to be visible only to the user 18 and information of said type can be prevented from being visible to unauthorized third parties.

The item of examination-relevant information or the plurality of items of examination-relevant information can also comprise status information relating to the magnetic resonance device 12. For example, the status information can comprise an examination protocol, in particular a timetable, of the magnetic resonance device 12 so that on the basis of the status information it is possible to estimate a time when the medical examination, in particular the magnetic resonance examination, to be conducted on the patient 17 will begin or alternatively how long the medical examination, in particular the magnetic resonance examination, to be conducted on the patient 17 still has to run.

The item of examination-relevant information or the plurality of items of examination-relevant information can also comprise information relating to a termination of the medical examination. In this way the user 18 can already obtain information that enables further planning, in particular a further plan in relation to further examinations to be conducted on the patient 17 and/or further plans in relation to the medical imaging device 11.

In a method step 103 following on therefrom, the examination-relevant information is visualized for the user 18 by means of the mobile data glasses 19. In this case the examination-relevant information is displayed on and/or projected onto a side of a lens 25 facing toward the user 18 of the mobile data glasses 19 (FIGS. 3 and 4). For this purpose the communication unit 22 has a visualization unit 28 that displays and/or projects the examination-relevant information on the lens 25 of the mobile data glasses 19. This enables a visualization in a field of view and/or a focus range of the user 18. The visualization unit can in this case be a projection unit and/or the visualization unit 24 can comprise the lens 25, which can be embodied as a visualization surface and/or as a screen surface.

In this method step 103, an acknowledgment of a display or information to the effect that an operating step has already been completed and a next item of examination-relevant information can be displayed can be entered by the user 18. An input of this type by the user 18 can be realized by way of a voice input or by way of an eye-tracking method. In principle, other input methods deemed beneficial by the person skilled in the art are also possible at any time.

FIG. 5 illustrates an alternative exemplary embodiment of the method for communication between a medical network 10 into which at least one medical imaging device 11 is integrated and a user 18 using mobile data glasses 19. Components, features and functions that remain substantially the same are labeled consistently with the same reference numerals. The following description is limited essentially to the differences compared to the exemplary embodiment in FIG. 3, with reference being made to the description of the exemplary embodiment in FIG. 3 in respect of components, features and functions that remain the same.

The method steps 100, 101 of connecting the mobile data glasses 19 to the medical network 10 and of selecting a medical examination to be conducted on a patient 17 from the database 13 by means of the communication unit 22 are performed analogously to the description of the method steps 100, 101 relating to FIG. 3.

In a method step 200 following on from the method step 101, environmental data of the user 18 is acquired by means of an acquisition unit 29 of the mobile data glasses 19 (FIG. 2). For this purpose the acquisition unit 29 has a camera which is arranged inside the mobile data glasses 29. The environmental data of the user 18 advantageously comprises a field of view and/or a visual range, in particular a focus range, of the user 18. For example, the environmental data of the user 18 concerns a patient 17 prior to the medical examination or the patient 17 during the medical examination. Preferably, the acquired data of the sensor unit 24 is also used for detecting the field of view, in particular the focus range, of the user 18. The acquisition of the environmental data of the user 18 enables an automated acquisition of information and/or detection of situations which enable an automated evaluation and consequently reduces and/or prevents a subjective evaluation and/or assessment of a situation by the user 18.

In a further method step 201, at least one item of examination-relevant information is generated by the communication unit 22 on the basis of the acquired environmental data of the user 201. The examination-relevant information is in this case dependent on the acquired environmental data and consequently the examination-relevant information can be generated in the context of the acquired environmental data. In particular, the one item of examination-relevant information or the plurality of items of examination-relevant information is or are generated as a function of the detected field of view, in particular of the focus range, of the user 18. In this case the examination-relevant information is generated automatically and/or autonomously by the communication unit 22 of the mobile data glasses 19.

The item of examination-relevant information or the plurality of items of examination-relevant information can comprise safety information. For example, the safety information can concern loose-hanging cables, which can lead to the cables being damaged when the patient 17 is introduced into a patient receiving zone 15 of a medical imaging device 11. Furthermore, the safety information can for example also concern uninserted plug-in connectors for ancillary units, for example an ECG unit and/or an injection unit, which are required for the medical examination. Moreover, the safety information can also comprise patient-specific safety information, such as an arranging of the extremities of the patient 17 to form a closed loop, for example, which in the case of a magnetic resonance examination can lead to an undesirable heating of the patient 17.

The item of examination-relevant information or the multiple items of examination-relevant information can in this case be at least one aid and/or at least one working instruction for the selected medical examination, in particular the magnetic resonance examination, to be conducted on the patient 17. The at least one aid and/or the at least one working instruction can for example comprise positioning information for a local radiofrequency antenna unit for a medical imaging examination to be conducted on the patient 17 during a positioning operation of a local radiofrequency antenna unit (see FIG. 4). Moreover, the at least one aid and/or the at least one working instruction can be a selection of the local radiofrequency antenna unit if, for example, no selection of a local radiofrequency antenna unit has yet been made by the user 18 for the pending magnetic resonance examination.

The item of examination-relevant information or the multiple items of examination-relevant information can also be patient-specific information, as has already been explained in more detail in relation to the description with reference to FIG. 3. Furthermore, the item of examination-relevant information or the plurality of items of examination-relevant information can also comprise status information relating to the magnetic resonance device 12, as has already been explained in more detail in relation to the description with reference to FIG. 3. Moreover, the item of examination-relevant information or the multiple items of examination-relevant information can also be information relating to a termination of the medical examination, as has already been explained in more detail in relation to the description with reference to FIG. 3.

In a further method step 202, the examination-relevant information is presented to the user 18 by the mobile data glasses 19, in particular by the visualization unit 28 of the mobile data glasses 19, analogously to the description with reference to FIG. 3. A context-dependent output of information to the user 18 is realized by means of the acquisition of environmental data. For example, in this way the user 18 can be alerted to acute and/or real risks to the patient 17. Moreover, working instructions and/or aids that are displayed to the user 18 by means of the mobile data glasses 19 can also be adapted with respect to a current situation of the patient 17 and/or of the user 18.

Although modifications and changes may be suggested by those skilled in the art, it is the intention of the inventor to embody within the patent warranted hereon all changes and modifications as reasonably and properly come within the scope of his contribution to the art.

We claim as our invention:

1. A method for communication between a medical network into which at least one medical imaging apparatus is integrated, and a user situated at an examination site at which a medical examination is conducted with the medical imaging apparatus, said method comprising:
providing mobile data glasses to the user, said mobile data glasses being worn by the user at said examination site, said mobile data glasses comprising a communication processor;
placing said communication processor of said mobile data glasses in communication with said medical network via at least one interface;
via said communication processor, accessing a database of the network and selecting, from said database, said medical examination to be conducted on a patient at said examination site;
also via said communication processor of said mobile data glasses, selecting at least one item of examination information associated with conducting said medical examination; and
via said communication processor, causing display of said at least one item of examination information at said mobile data glasses so as to be visible by the user.

2. A method as claimed in claim 1 comprising communicating wirelessly between said mobile data glasses and said medical network.

3. A method as claimed in claim 1 wherein said communication unit is configured to automatically select said medical examination.

4. A method as claimed in claim 1 wherein said communication processor is configured to receive an input made by the user that selects said medical examination.

5. A method as claimed in claim 1 wherein said communication processor is configured to select a medical imaging examination as said medical examination to be conducted at said examination site on the patient.

6. A method as claimed in claim 1 wherein said communication processor is configured to automatically select said at least one item of examination information dependent on the selected medical examination to be conducted at the examination site on the patient.

7. A method as claimed in claim 1 wherein said at least one item of examination information is selected from the group consisting of an aid to conducting said medical examination and a working instruction for conducting said medical examination.

8. A method as claimed in claim 1 wherein said at least one item of examination information comprises patient-specific information that is specific to the patient.

9. A method as claimed in claim 1 wherein said at least one item of examination information comprises status information about said medical imaging apparatus.

10. A method as claimed in claim 1 wherein said at least one item of examination information comprises information relating to termination of said medical examination.

11. A method as claimed in claim 1 wherein said mobile data glasses comprise an environmental data acquisition detector, and comprising detecting environmental data of the user with said environmental data acquisition detector at said mobile data glasses.

12. A method as claimed in claim 11 wherein said communication processor is configured to select said at least one item of examination information dependent on the acquired environmental data.

13. A method as claimed in claim 1 wherein said at least one item of examination information comprises safety information.

14. A method as claimed in claim 1 comprising a detector at said mobile data glasses that detects a field of view of the user through said mobile data glasses, and wherein said communication processors configured to select said at least one item of examination information dependent on the detected field of view.

15. Mobile data glasses adapted to be worn by a user performing a medical examination at a medical imaging apparatus that is in communication with a medical network, said mobile data glasses comprising:
a communication processor;
at least one interface that places said communication processor in communication with said medical network via at least one interface;
said communication processor being configured to access a database of the network and select, from said database, said medical examination to be conducted on a patient at said examination site;
said communication processor being configured to also select at least one item of examination information associated with conducting said medical examination;
a display; and
said communication processor being configured to cause display of said at least one item of examination information at said display so as to be visible by the user.

16. Mobile data glasses as claimed in claimed 15 comprising a detector that detects environmental data of the user, and wherein said communication processor is configured to select said at least one item of examination information dependent on the detected environmental data.

17. A non-transitory, computer-readable data storage medium encoded with programming instructions, said storage medium being distributively loaded into a communication processor of mobile data glasses worn by a user at an examination site for conducting a medical examination on a patient using a medical imaging apparatus that is in communication with a network, said programming instructions causing said communication processor to:
place said communication processor of said mobile data glasses in communication with said medical network via at least one interface;
access a database of the network and select, from said database, said medical examination to be conducted on a patient at said examination site;
also select at least one item of examination information associated with conducting said medical examination; and
cause display of said at least one item of examination information at said mobile data glasses so as to be visible by the user.
